(19) United States
(12) Patent Application Publication
(10) Pub. No.: US 2024/0041893 A1
(43) Pub. Date: Feb. 8, 2024

(54) An Extended-Release Pharmaceutical Formulation of Viloxazine And Process For Preparation Thereof

(71) Applicant: Innovate Therapeutics LLC, Princeton, NJ (US)

(72) Inventor: Jayendrakumar Dasharathlal Patel, Plainsboro, NJ (US)

(73) Assignee: Innovate Therapeutics LLC, Princeton, NJ (US)

(21) Appl. No.: 18/479,990

(22) Filed: Oct. 3, 2023

(51) Int. Cl. A61K 31/5375; A61K 9/16

(52) U.S. Cl. CPC A61K 31/5375; A61K 9/167

(57) ABSTRACT

A pharmaceutical formulation comprising a single population of IR/XR particles of viloxazine, wherein each IR/XR particle of the single population of IR/XR particles comprising an extended-release region (XR) of the viloxazine; and an immediate-release region (IR) of the viloxazine, wherein the immediate-release region (IR) of the viloxazine is coated surrounding the extended-release region (XR) of the viloxazine, wherein the extended-release region (XR) is formed as an extended-release particle and the immediate-release region (IR) is formed as a coat surrounding the extended-release particle. A process for making the single population of IR/XR particles of viloxazine comprising the steps in the sequence of: (a) making the extended-release (XR) particles of the viloxazine; and (b) making the immediate-release (IR) coat of the viloxazine surrounding the extended-release (XR) particles of the viloxazine.

### FIELD OF THE DISCLOSURE

The disclosure relates generally to a pharmaceutical formulation of viloxazine formulated in a single population of IR/XR-particles, and the related method of making the pharmaceutical formulation and treatment of the disease condition using the pharmaceutical formulation of the viloxazine.

### BACKGROUND

Viloxazine ((R,S)-2-[(2-ethoxyphenoxy)methyl]morpholine]) is a bicyclic morpholine derivative. It is characterized by the formula Ci 3 Hi 9 NO 3. Viloxazine has two stereoisomers, (S)-(−)- and (R)-(+)-isomer, which have the following chemical structures:

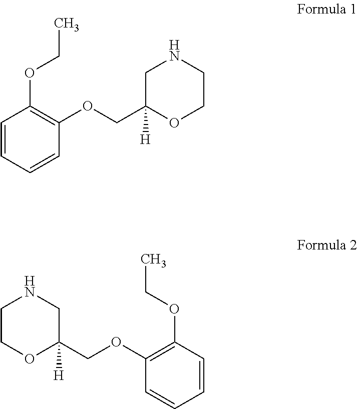 [SMILES: CCOc1ccccc1OC[C@H]1CNCCO1 ; CCOc1ccccc1OC[C@@H]1CNCCO1]

Viloxazine is known to have several desirable pharmacologic uses, including treatment of depression, nocturnal enuresis, narcolepsy, sleep disorders, and alcoholism, among others. In vivo, viloxazine acts as a selective norepinephrine reuptake inhibitor (“NRI”). Between the two stereoisomers, the (S)-(−)-isomer is known to be five times as pharmacologically active as the (R)-(+)-isomer. Viloxazine hydrochloride, (±)-2-[(2-ethoxyphenoxy)methyl]morpholine hydrochloride [Structural Formula 1, a racemic compound with two stereoisomers (R-viloxazine and S-viloxazine] has a molecular weight of 273.8 with a conversion factor for viloxazine base to viloxazine hydrochloride of 1.154. The typical “immediate release” oral dose of viloxazine in MDD, expressed as viloxazine base, was 200 mg-300 mg daily given in 2 to 3 divided doses; in certain cases, the daily dose was increased to 600 mg (Vidal® pp 2116-2117 (2007)). The absolute oral bioavailability was 85% (±14%, standard deviation) (E. Pisani et al. Psychopharmacology (1986) 90: 295-298). The drug was rapidly absorbed following oral administration with a t max of approximately 2 hours. The observed elimination half-life was 4.3 hours.

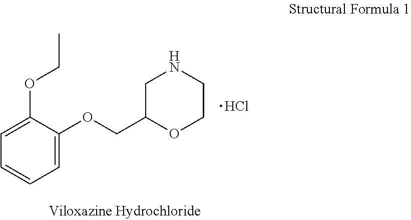

Due to the potentially high therapeutic dose, weakly basic nature of the molecule, and a relatively high in vivo clearance rate in humans, viloxazine presents challenges for developing an extended-release formulation. The US patents U.S. Pat. No. 9,662,338B2, U.S. Pat. No. 9,603,853B2, and U.S. Pat. No. 9,358,204B2, assigned to Super Pharmaceuticals Inc, disclosed various approaches for manufacturing the extended-release pharmaceutical formulation of the viloxazine, which includes matrix system, drug-layered system, osmotic release system, and gastro-retentive system. The prior art (U.S. Pat. No. 9,662,338B2, U.S. Pat. No. 9,603,853B2, and U.S. Pat. No. 9,358,204B2) disclosed various formulation approaches based on a mixed population of particles and a single population of particles.

Various disclosed formulation approaches based on the mixed population of particles include mixed IR and XR particles where individually prepared IR particles are mixed with individually prepared XR particles (IR/XR mixed particles), mixed XR particles where two individually prepared XR particles having differing drug release characteristics are combined (mixed XR particles), mixed DR particles where two individually prepared DR particles of differing drug release characteristics are combined (mixed DR particles), mixed IR and DR particles where individually prepared IR particles are mixed with individually prepared DR particles (IR/DR mixed particles), mixed IR and DR-XR particles where individually prepared IR particles are mixed with individually prepared DR coated XR particles (IR/DR-XR particles), and mixed XR and DR particles where individually prepared XR particles are mixed with individually prepared DR particles (XR/DR mixed particles).

Various disclosed formulation approaches based on the single population of particles formulation approaches include matrix XR particles (formulating a single population of XR particles), coated XR particles (where IR cores are coated with XR coating to formulate the single population of XR particles), IR cores are coated with DR coat and further coated with IR coat (formulating a single population of IR/DR particles), IR cores coated first with XR coat, then coated with DR coat and lastly coated with IR coat (formulating a single population of IR/DR-XR particles), and IR cores coated first with DR coat, then coated with drug layer (drug coat), and lastly coated with XR coat (formulating a single population of XR-f/DR particles).

While the prior art (U.S. Pat. No. 9,662,338B2, U.S. Pat. No. 9,603,853B2, and U.S. Pat. No. 9,358,204B2) disclosed formulating a single population of XR particles, IR/DR particles, IR/DR-XR particles, XR-f/DR particles, it does not disclose formulation of a single population of IR/XR particles. Although, the prior art (U.S. Pat. No. 9,662,338B2, U.S. Pat. No. 9,603,853B2, and U.S. Pat. No. 9,358,204B2) disclosed formulating the mixed population of IR/XR particles by simply mixing two individually prepared particles (components), i.e., mixing the individually prepared IR particles with the individually prepared XR particles. However, formulating the mixed population of IR/XR particles necessitates additional unit operations (such as preparation of two individual particles, i.e., IR particles and XR particles, blending IR particles with XR particles in an appropriate ratio prior to the capsule filling process) and may also necessitating a tedious, complex two-station capsule filling machine that can fill the first XR particles at the first station and the second IR particles at the second station, or vice versa.

The present subject matter disclosed a pharmaceutical formulation formulated in a single population of IR/XR-particles of viloxazine, wherein the immediate-release (IR) dose of the viloxazine and the extended-release (XR) dose of the viloxazine is contained within same particle (each particle) of the single population of IR/XR-particles of the viloxazine, wherein the extended-release (XR) dose is incorporated by formulating XR particles and the immediate-release (IR) dose is incorporated by formulating an immediate-release coat(s) of the viloxazine surrounding the XR particles. Compared to the mixed population of IR/XR particles as disclosed in the prior art, the presently disclosed single population of IR/XR particles of the viloxazine can be filled in capsules using the single station conventional capsule filling machine at high speed, thus eliminating the requirement of the tedious, complex two-station capsule filling machine, and also eliminate the blending unit operation for mixing two distinct types of particles having different release characteristics, i.e., IR particles and XR particles, thus reducing the risk of blend uniformity (issue of getting uniformity in mixing of XR particles with IR particles).

### SUMMARY

In certain embodiments, a pharmaceutical formulation comprising a single population of IR/XR-particles of viloxazine, wherein each IR/XR-particle of the single population of IR/XR-particles comprising an immediate-release (IR) region of viloxazine; and an extended-release (XR) region of viloxazine, wherein the extended-release (XR) region of viloxazine is included in form of an XR-particle, and the immediate-release (IR) region of viloxazine is included in form of an immediate-release coat (layer) surrounding the XR particle of the viloxazine, wherein a weight ratio of the total weight of the XR-particles components and the total weight of the immediate-release coat components is in a range of about 1.5:1 to about 50:1, respectively, wherein a weight ratio of the viloxazine present in the extended-release (XR) region and the immediate-release (IR) region is in a range of about 0.25:1 to about 25:1, respectively. In certain embodiments, the XR particle comprising the viloxazine embedded within a matrix of at least one release rate-controlling ingredient, wherein the matrix is in the form of a matrix particle or a matrix coat surrounding an inert core, wherein the matrix, optionally, further comprising one or more pharmaceutically acceptable excipients, and is, optionally, further coated with a release rate controlling coat (RC) of at least one release rate-controlling ingredient. In certain embodiments, the XR particle comprising a release rate-controlling (RC) coat of at least one release rate-controlling ingredient surrounding a core particle of the viloxazine, wherein the release rate-controlling (RC) and the core particle, optionally, further comprising one or more pharmaceutically acceptable excipients. In certain embodiments, the immediate-release coat comprising the viloxazine, and, optionally, one or more pharmaceutically acceptable excipients. In certain embodiments, an amount of the viloxazine contained in the immediate-release coat is not less than about 5%, preferably not less than about 15%, more preferably not less than about 35%, and most preferably not less than about 50% w/w of the total weight of the immediate-release coating components, wherein the immediate-release coating components include the viloxazine, and, optionally, the one or more pharmaceutically acceptable excipients.

In certain embodiments, the immediate-release coat(s) or region(s) of the single population of IR/XR-particles releases the viloxazine in an amount of more than about 75% of the total weight of the viloxazine contained in the immediate-release coat(s) or region(s) in less than about 1 hour when measured in 900 ml of an aqueous medium (water, 0.1N HCl, acetate buffer pH 4.5 and phosphate buffer pH 6.8) at a temperature of 37°±1° C. using either USP apparatus 2 (paddle) at 50 RPM (or 75 RPM) or USP apparatus 1 (basket) at 100 RPM (or 150 RPM).

In certain embodiments, the extended-release (XR) particle(s) or region(s) of the single population of IR/XR-particles releases the viloxazine in an amount of less than about 75% of the total weight of the viloxazine contained in the extended-release (XR) particle(s) or region(s) in less than about 2 hours (120 minutes) when measured in 900 ml of an aqueous medium (water, HCl, acetate buffer pH 4.5 and phosphate buffer pH 6.8) at a temperature of 37°±1° C. using either USP apparatus 2 (paddle) at 50 RPM (or 75 RPM) or USP apparatus 1 (basket) at 100 RPM

In certain embodiments, the release characteristics (release rate) of the viloxazine from the extended-release (XR) particle(s) or region(s) of the single population of IR/XR-particles are substantially resistant to alcohol dose dumping, wherein the extended-release (XR) particle(s) or region(s) of the single population of IR/XR-particles comprises a mixture of at least two release rate-controlling ingredient, wherein the at least one release rate-controlling ingredient is a water-insoluble ingredient and the at least another release rate-controlling ingredient is a water-soluble ingredient, wherein a % weight ratio of the water-insoluble ingredient to the water-soluble ingredient is any from about 20:80 to about 99.9:0.1, preferably from about 40:60 to about 99:1, more preferably from about 50:50 to about 96:4, and most preferable from about 60:40 to about 90:10, respectively. However, the % weight ratio of the water-insoluble ingredient to the water-soluble ingredient varies depending on the selection of the grade of ingredient. For example, ethyl cellulose:guar gum (very low viscosity grade of guar gum) in a ratio of 90:10 is not effective to resist alcohol dose dumping, but ethyl cellulose:guar gum (high viscosity grade of the guar gum) in the same ratio of 90:10 at the same coating weight gain is highly effective to prevent alcohol dose dumping (reference incorporated herein completely: E Rosiaux, S. Muschert, R. Chokshi, B. Leclercq, F. Siepmann, J. Siepmann, Ethanol-resistant polymeric film coatings for controlled drug delivery, Journal of Controlled Release, Volume 169, Issues 1-2, 2013, Pages 1-9).

In certain embodiments, a process for making a single population of IR/XR particles of viloxazine comprising the steps in the sequence of: (a) first making the extended-release (XR) particles of the viloxazine; and (b) second making the immediate-release (IR) coat of the viloxazine surrounding the extended-release (XR) particles of the viloxazine.

In certain embodiments, each extended-release particle (XR-particle) or region of the single population of IR/XR particles of viloxazine is prepared by embedding or dispersing the viloxazine within a matrix of the at least one release rate-controlling ingredient, wherein the matrix is prepared in the form of a matrix particle or a matrix coat. In certain embodiments, a mixture of the viloxazine with the at least one release rate-controlling ingredient forms the matrix particles or the matrix coats that are capable of limiting the dissolution rate of the viloxazine from the extended-release particle (XR-particle) into an aqueous medium. The matrix material (such as the at least one release rate-controlling ingredient) useful for these embodiments are generally water-insoluble materials, slowly water-soluble material, or a combination of water-insoluble materials and water-soluble materials. If needed, the matrix material(s) may optionally be formulated with water-soluble materials, which can be used as binders or as permeability-modifying agents in the matrix. In certain embodiments, the prepared XR-particles are further coated with an optional release rate controlling coat, wherein the release rate controlling coat comprises water-insoluble materials, slowly water-soluble material, or a combination of water-insoluble materials, and water-soluble materials.

In certain embodiments, each extended-release particle (XR-particle) or region of the single population of IR/XR particles of viloxazine is prepared by applying a release rate-controlling (RC) coat of the at least one release rate-controlling ingredient surrounding a core particle of the viloxazine, wherein the release rate-controlling (RC) coat, optionally, further comprising the one or more pharmaceutically acceptable excipients. In certain embodiments, the applied release rate-controlling (RC) coat surrounding the core particles of the viloxazine is capable of limiting the dissolution rate of the viloxazine from the extended-release particles (XR-particles) into an aqueous medium. The release rate-controlling ingredients useful to prepare the release rate-controlling coat are generally water-insoluble materials, slowly water-soluble materials, or a combination of water-insoluble materials and water-soluble materials. If needed, the water-soluble materials in an appropriate amount can be added as permeability-modifying agents in the release rate-controlling coat to achieve the desired extended-release profile of viloxazine. In certain embodiments, the viloxazine can also be used as a soluble material or permeability-modifying agent in the release rate controlling coat to achieve the desired extended-release profile of viloxazine. In certain embodiments, the prepared XR-particles are, optionally, further coated with a second release rate controlling coat, wherein the second release rate controlling coat comprises water-insoluble materials, slowly water-soluble material, or a combination of water-insoluble materials, and water-soluble materials. In certain embodiments, the core particle of the viloxazine comprises the viloxazine, and optionally, at least one or more pharmaceutically acceptable excipients, wherein the core particle of the viloxazine can be prepared, formulated or used in the form of (a) a particle of the viloxazine (a pure drug substance); (b) a matrix particle of the viloxazine, wherein the viloxazine is embedded within a matrix of the one or more pharmaceutically acceptable excipient; or (c) a coat of the viloxazine surrounding an inert core.

In certain embodiments, the immediate release coat or region of viloxazine is included surrounding each XR-particle of the single population of IR/XR particles of viloxazine. A most preferred process for manufacturing the immediate release coat or region of viloxazine is: dissolve the viloxazine, and optionally, one or more pharmaceutical-acceptable excipients in enough amount of solvent (aqueous, organic, or mixture thereof), and spray the resulting solution on the prepared XR-particles of viloxazine to form the immediate release coat or region of viloxazine surrounding each XR-particle of the single population of IR/XR particles of viloxazine. The conventional pan coating or Wurster coating process can be used to prepare the immediate release coat or region of viloxazine. A further useful process for preparing the immediate release coat or region of viloxazine is powder layering of the viloxazine or a mixture of the viloxazine and one or more pharmaceutical-acceptable excipients surrounding the XR-particles of viloxazine to form the immediate release coat or region of viloxazine surrounding each XR-particle of the single population of IR/XR particles of viloxazine.

### DETAILED DESCRIPTION

The terms used in this specification generally have their ordinary meanings in the art, within the context of this present disclosure, and in the specific context where each term is used. Certain terms are discussed below, or elsewhere in the specification, to provide additional guidance to the practitioner in describing the formulation and how to make and use them.

As used herein, the use of the word “a” or “an” when used in conjunction with the term “comprising” in the claims and the specification can mean “one,” but it is also consistent with the meaning of “one or more,” “at least one,” and “one or more than one.” Still further, the terms “having,” “including,” “containing”, “comprising” and “comprises” are interchangeable, and one of skill in the art is cognizant that these terms are open-ended terms.

As used in this specification, the term “pharmaceutical formulation” refers to an oral pharmaceutical dosage form formulated as any of a solid oral dosage form and a liquid oral dosage form for oral administration, preferably, peroral administration, and comprising a therapeutically effective amount of viloxazine (eg. from about 10 mg to about 1000 mg of viloxazine).

The term “drug”, “drug substance”, “active pharmaceutical agent”, “active agent”, “active pharmaceutical ingredient”, and “active ingredient” refers to “Viloxazine”. The term “Viloxazine,” means (RS)-2-[(2-ethoxyphenoxy)methyl]morpholine or a pharmaceutically acceptable salt or ester thereof, any polymorph thereof as well as variable mixtures of the R and S enantiomers or either one of the R or S enantiomers in a substantially pure form. Further, “viloxazine” can be employed in the pharmaceutical formulation in any form including, but not limited to, in base form (as such), salt form (such as Viloxazine Hydrochloride), any pharmaceutically acceptable form, any polymorphic form, anhydrous as well as hydrated form, solvates, co-crystal, crystalline or amorphous form, a single-component or multiple-component crystal, clathrate, etc. All such forms of “viloxazine” are within the scope of the present disclosure.

In the term “a single population of IR/XR particles of viloxazine”, the letter “IR” refers to immediate-release, and XR refers to extended-release.

The term “about” or “approximately” means within an acceptable error range for the particular value as determined by one of ordinary skill in the art. For example, “about” can mean a range of up to 20%, up to 15%, up to 10%, up to 5%, or up to 1% of a given value.

The term “immediate release” refers to a release characteristic of “viloxazine” contained in an “immediate release region(s) or coat(s)” of the presently disclosed pharmaceutical formulation that releases the “viloxazine” in an amount of more than about 75% w/w of the total weight of the “viloxazine” contained within such region(s) or coat(s) in less than about 1 hour when measured in 900 ml of an aqueous medium (water, 0.1N HCl, acetate buffer pH 4.5 and phosphate buffer pH 6.8) at a temperature of 37°±1° C. using either USP apparatus 2 (paddle) at 50 RPM (or 75 RPM) or USP apparatus 1 (basket) at 100 RPM (or 150 RPM). The term “immediate release (IR) region(s) or coat(s)” refers to region(s) or coat(s) included in a pharmaceutical formulation where the majority amount of the “viloxazine” (more than about 75% by weight of the “viloxazine”) present within such region(s) or coat(s) is released in less than about 1 hour when measured in 900 ml of an aqueous medium (water, 0.1N HCl, acetate buffer pH 4.5 and phosphate buffer pH 6.8) at a temperature of 37°±1° C. using either USP apparatus 2 (paddle) at 50 RPM (or 75 RPM) or USP apparatus 1 (basket) at 100 RPM (or 150 RPM). The immediate release characteristics can be determined by performing the dissolution on placebo XR-particles (viloxazine-free XR-particles) coated with an immediate-release drug coat of viloxazine.

An “extended release” refers to a release characteristic of “viloxazine” contained in “an extended-release (XR) particle(s) or region(s)” of the presently disclosed pharmaceutical formulation that releases the “viloxazine” in an amount of not more than about 75% of the total weight of the “viloxazine” contained within such particle(s) or region(s) in less than about 2 hours when measured in 900 ml of an aqueous medium (water, 0.1N HCl, acetate buffer pH 4.5 and phosphate buffer pH 6.8) at a temperature of 37°±1° C. using either USP apparatus 2 (paddle) at 50 RPM (or 75 RPM) or USP apparatus 1 (basket) at 100 RPM (or 150 RPM). The term “extended-release (XR) particle(s) or region(s)” refers to particle(s) or region(s) included in a pharmaceutical formulation where the majority amount of the “viloxazine” (more than about 75% by weight of the “viloxazine”) present within such region is not released in less than about 2 hours when measured in 900 ml of an aqueous medium (water, 0.1N HCl, acetate buffer pH 4.5 and phosphate buffer pH 6.8) at a temperature of 37°±1° C. using either USP apparatus 2 (paddle) at 50 RPM (or 75 RPM) or USP apparatus 1 (basket) at 100 RPM (or 150 RPM). The extended-release characteristics can be determined by performing dissolution on plain XR-particles of viloxazine, (XR-particles of viloxazine prior to applying an immediate-release drug coat of viloxazine).

The term “immediate-release (IR) coat(s) components” or “immediate-release region(s) components” refers to all ingredients, including drug; and, optional, pharmaceutically acceptable ingredients, used to prepare immediate-release (IR) coat(s) or the immediate-release region(s).

The term “XR-particles components” or “the extended-release region(s) components” refers to all ingredients, including drug; release rate controlling ingredient; and, optional, pharmaceutically acceptable ingredients, used to prepare XR-particles or the extended-release region(s).

The term “particles” or “particulates”, as used herein, includes, without any limitations on the nature and size thereof, any particles, spheres, beads, granules, pellets, mini-tablets, mini-capsules, and particulates. The particles generally are of a diameter or length from about 5 microns to about 2.5 cm, with a preferred range of 100 microns to 2.5 mm.

The term “core particle” refers to the internal foundation of a structural unit (e.g., particle, sphere, bead, granule, pellet, mini-tablet, mini-capsule, and particulate) with or without a drug.

The term “pharmaceutically acceptable excipient” refers to those ingredients that are well accepted by the industry and regulatory agencies such as those listed in monographs published in compendia such as USP-NF, Food Chemicals Codex, Code of Federal Regulations (CFR), FDA Inactive Ingredients Guide and in 21 CFR parts 182 and 184 that lists substances that are generally regarded as safe (GRAS) food ingredients.

The term “hydro-alcoholic mixture or solution” refers to a mixture or solution containing up to 40% v/v ethanol in the overall volume of water or any aqueous media, based upon the total weight of the mixture or solution.

The term “high drug load” used in the current application that applies to the pharmaceutical formulations comprising the viloxazine in an amount at least about 35% w/w of the total weight of the pharmaceutical formulation.

In certain embodiments, a pharmaceutical formulation comprising a single population of IR/XR-particles of viloxazine, wherein each IR/XR-particle of the single population of IR/XR-particles comprising an immediate-release (IR) region of viloxazine; and an extended-release (XR) region of viloxazine, wherein the extended-release (XR) region of viloxazine is included in form of an XR-particle, and the immediate-release (IR) region of viloxazine is included in form of an immediate-release coat (layer) surrounding the XR particle of the viloxazine, wherein a weight ratio of the total weight of the XR-particle components and the total weight of the immediate-release coat components is in a range of about 1.5:1 to about 50:1, respectively, wherein a weight ratio of the viloxazine present in the extended-release (XR) region and the immediate-release (IR) region is in a range of about 0.25:1 to about 25:1, respectively. In certain embodiments, the XR particle comprising the viloxazine embedded within a matrix of at least one release rate-controlling ingredient, wherein the matrix is in the form of a matrix particle or a matrix coat surrounding an inert core, wherein the matrix, optionally, further comprising one or more pharmaceutically acceptable excipients, and is, optionally, further coated with a release rate controlling coat (RC) of at least one release rate-controlling ingredient. In certain embodiments, the XR particle comprising a release rate-controlling (RC) coat of at least one release rate-controlling ingredient surrounding a core particle of the viloxazine, wherein the release rate-controlling (RC) and the core particle, optionally, further comprising one or more pharmaceutically acceptable excipients. In certain embodiments, the immediate-release coat comprising the viloxazine, and, optionally, one or more pharmaceutically acceptable excipients. In certain embodiments, an amount of the viloxazine contained in the immediate-release coat is not less than about 5%, preferably not less than about 15%, more preferably not less than about 35%, and most preferably not less than about 50% w/w of the total weight of the immediate-release coating components, wherein the immediate-release coating components include the viloxazine, and, optionally, the one or more pharmaceutically acceptable excipients.

In certain embodiments, a pharmaceutical formulation comprising a single population of IR/XR particles of viloxazine,
wherein each IR/XR particle of the single population of IR/XR particles comprising an extended-release region (XR) of the viloxazine; and an immediate-release region (IR) of the viloxazine, wherein the immediate-release region (IR) of the viloxazine is coated surrounding the extended-release region (XR) of the viloxazine,
   wherein the extended-release region (XR) is formed as an extended-release particle,
   wherein the extended-release particle comprising
      the viloxazine that is embedded within a matrix of at least one release rate-controlling ingredient, wherein the matrix is in the form of a matrix particle or a matrix coat surrounding an inert core, wherein the matrix, optionally, further comprising one or more pharmaceutically acceptable excipients, and is, optionally, further coated with a release rate controlling coat (RC) of the at least one release rate-controlling ingredient;
         or
      a release rate-controlling (RC) coat of at least one release rate-controlling ingredient surrounding a core particle of the viloxazine, wherein at least one of the release rate-controlling coat (RC) and the core particle, optionally, further comprising one or more pharmaceutically acceptable excipients,
   wherein the immediate-release region (IR) is formed as a coat surrounding the extended-release particle, and comprising the viloxazine; and, optionally, the one or more pharmaceutically acceptable excipients,
wherein a weight ratio of the total weight of the extended-release region (XR) components and the total weight of the immediate-release region (IR) components present in the single population of IR/XR particles of viloxazine is in a range of about 1.5:1 to about 50:1, respectively,
wherein a weight ratio of the viloxazine present in the extended-release region (XR) and the immediate-release region (IR) of the single population of IR/XR particles of viloxazine is in a range of about 0.25:1 to about 25:1, respectively, wherein the amount of the viloxazine contained in the immediate-release region is not less than about 5% w/w of the total weight of the immediate-release region (IR) components,
wherein a process for making the single population of IR/XR particles of viloxazine comprising the steps in the sequence of:
   (a) making the extended-release (XR) particles of the viloxazine; and
   (b) making the immediate-release (IR) coat of the viloxazine surrounding the extended-release (XR) particles of the viloxazine.

In certain embodiments, the immediate-release coat(s) or region(s) of the single population of IR/XR-particles releases the viloxazine in an amount of more than about 75% of the total weight of the viloxazine contained in the immediate-release coat(s) or region(s) in less than about 1 hour when measured in 900 ml of an aqueous medium (water, 0.1N HCl, acetate buffer pH 4.5 and phosphate buffer pH 6.8) at a temperature of 37°±1° C. using either USP apparatus 2 (paddle) at 50 RPM (or 75 RPM) or USP apparatus 1 (basket) at 100 RPM (or 150 RPM).

In certain embodiments, the extended-release (XR) particle(s) or region(s) of the single population of IR/XR-particles releases the viloxazine in an amount of less than about 25% of the total weight of the viloxazine contained in the extended-release (XR) particle(s) or region(s) in less than about 0.5 hour (30 minutes) when measured in 900 ml of an aqueous medium (water, 0.1N HCl, acetate buffer pH 4.5 and phosphate buffer pH 6.8) at a temperature of 37°±1° C. using either USP apparatus 2 (paddle) at 50 RPM (or 75 RPM) or USP apparatus 1 (basket) at 100 RPM

In certain embodiments, the extended-release (XR) particle(s) or region(s) of the single population of IR/XR-particles releases the viloxazine in an amount of less than about 50% of the total weight of the viloxazine contained in the extended-release (XR) particle(s) or region(s) in less than about 1 hour (60 minutes) when measured in 900 ml of an aqueous medium (water, 0.1N HCl, acetate buffer pH 4.5 and phosphate buffer pH 6.8) at a temperature of 37°±1° C. using either USP apparatus 2 (paddle) at 50 RPM (or 75 RPM) or USP apparatus 1 (basket) at 100 RPM (or 150 RPM).

In certain embodiments, the extended-release (XR) particle(s) or region(s) of the single population of IR/XR-particles releases the viloxazine in an amount of less than about 75% of the total weight of the viloxazine contained in the extended-release (XR) particle(s) or region(s) in less than about 2 hours (120 minutes) when measured in 900 ml of an aqueous medium (water, HCl, acetate buffer pH 4.5 and phosphate buffer pH 6.8) at a temperature of 37°±1° C. using either USP apparatus 2 (paddle) at 50 RPM (or 75 RPM) or USP apparatus 1 (basket) at 100 RPM (or 150 RPM).

In certain embodiments, the extended-release (XR) particle(s) or region(s) of the single population of IR/XR-particles comprises the at least one release rate-controlling ingredient, wherein the at least one release rate-controlling ingredient is a water-insoluble ingredient. In certain embodiments, the extended-release (XR) particle(s) or region(s) of the single population of IR/XR-particles comprises a mixture of at least two release rate-controlling ingredients, wherein the at least one release rate-controlling ingredient is a water-insoluble ingredient and the at least another release rate-controlling ingredient is a water-soluble ingredient. In certain embodiments, the extended-release (XR) particle(s) or region(s) of the single population of IR/XR-particles comprises a mixture of at least two release rate-controlling ingredients, wherein the at least one release rate-controlling ingredient is a water-insoluble ingredient and the at least another release rate-controlling ingredient is a water-soluble ingredient, wherein a % weight ratio of the water-insoluble ingredient to the water-soluble ingredient is any from about 20:80 to about 99.9:0.1, preferably from about 40:60 to about 99:1, more preferably from about 50:50 to about 96:4, and most preferable from about 60:40 to about 90:10, respectively.

It is well known that extended-release pharmaceutical formulations are substantially sensitive to alcohol dose dumping when administered in the presence of ethanol. The term “dose dumping” as defined by the FDA, is the unintended, rapid release of a significant portion of a drug from a controlled release (extended-release) dosage form (Meyer, et al, “Awareness Topic: Mitigating the Risks of Ethanol Induced Dose Dumping From Oral Sustained/Controlled Release Dosage Forms,” FDA's ACPS Meeting, October, 2005). This phenomenon can, for example, be caused by the consumption of alcoholic beverages, leading to high ethanol concentrations in the contents of the stomach (Roth et al., “Ethanol Effects on Drug Release From Verapamil Meltrex, an Innovative Melt Extruded Formulation,” Int. J. Pharm., 368, 72-75, 2009). If drug release is extended by a polymer which is insoluble in water, but soluble in aqueous media containing significant amounts (40% v/v) of ethanol, the co-ingestion of alcoholic beverages can lead to the unintended dissolution of such polymer which controls the extended-release property of the drug. Thus, drug release can be rapid, instead of being extended over prolonged periods of time. For example, any extended-release pharmaceutical formulation comprising a release rate-controlling polymer such as ethyl cellulose that is water insoluble but soluble (fully, partially, or slowly) in alcohol (ethanol) or hydro-alcoholic mixture (up to 40% v/v ethanolic-water mixture) is highly sensitive to the dose dumping effect compared to an extended-release pharmaceutical formulation which comprises a release rate-controlling polymer (or ingredients) that is insoluble in alcohol (ethanol) or hydro-alcoholic mixture (up to 40% v/v ethanolic-water mixture) or comprises a mixture of two release rate-controlling ingredients, of which at least one release rate-controlling ingredient is insoluble in alcohol (ethanol) or hydro-alcoholic mixture (up to 40% v/v ethanolic-water mixture). Further, per the US FDA orange book information, the prior art patents U.S. Pat. No. 9,662,338B2, U.S. Pat. No. 9,603,853B2, and U.S. Pat. No. 9,358,204B2 assigned to “Super Pharmaceuticals Inc” disclosed the US FDA-approved viloxazine extended-release capsule that is marketed under the brand name of QELBREE™ (viloxazine extended-release capsule). Per the patents (U.S. Pat. No. 9,662,338B2, U.S. Pat. No. 9,603,853B2, and U.S. Pat. No. 9,358,204B2) information and list of ingredients mentioned on the approved product information label (PIL), it has been confirmed that the QELBREE™ (viloxazine extended-release capsule) comprises a mixture of ethyl cellulose (water insoluble but alcohol soluble) and Hypromellose (water-soluble but alcohol insoluble) as release rate controlling ingredients. It has been clearly mentioned in the “PRODUCT QUALITY REVIEW FILE(s)” of QELBREE™ (viloxazine extended-release capsule) submitted to the US FDA by NDA holder “Super Pharmaceuticals Inc” during the approval process that the currently approved “QELBREE™ (viloxazine extended-release capsule) has faster in-vitro alcohol dissolution in 20% and 40% ethanolic media compared to 0% ethanolic media. As mentioned on pages 54-58 of 67 in the “PRODUCT QUALITY REVIEW FILE(s)”, the QELBREE™ (viloxazine extended-release capsule) releases 100% viloxazine hydrochloride in 40% ethanolic media within 2 hours compared to the release of about 28% viloxazine hydrochloride in 0% ethanolic media within 2 hours [QELBREE™ (viloxazine extended-release capsule)—Center for Drug Evaluation & Research, Application Number 211964Orig1s000, Product Quality Review(s)—https://www.accessdata.fda.gov/drugsatfda_docs/nda/2021/211964Orig1s000ChemR.pdf]. Because the extended-release profile [of QELBREE™ (viloxazine extended-release capsule)] is not maintained in 20% and 40% ethanol, there may be a risk for dose dumping” which is further supported by comparative in-vivo pharmacokinetic data after administering QELBREE™ (viloxazine extended-release capsule) with and without alcoholic beverages, i.e., Tmax is reduced to about 2 hours (which is similar to immediate-release formulation of the viloxazine, and thus no beneficial effect of administering the extended-release formulation) after administering QELBREE™ (viloxazine extended-release capsule) with 40% alcohol contained in orange juice, compared to Tmax of about 5 hours when QELBREE™ (viloxazine extended-release capsule) administered with only orange juice, this phenomenon indicating alcohol-dose dumping.

Therefore, the selection of the right excipient, the right grade of the right excipient, and the right mixture of the right grade excipients in the right amount or ratio are also crucial in choosing the release rate-controlling ingredients to formulate the extended-release (XR) particle(s) or region(s) of the presently disclosed pharmaceutical formulation.

In certain embodiments, the release characteristics (release rate) of the viloxazine from the extended-release (XR) particle(s) or region(s) of the single population of IR/XR-particles is substantially resistant to alcohol dose dumping, wherein the extended-release (XR) particle(s) or region(s) of the single population of IR/XR-particles comprises the at least one release rate-controlling ingredient that is insoluble in at least one of water, an alcohol (ethanol), and a hydro-alcoholic mixture/solution (up to 40% v/v ethanolic-water mixture/solution). The term “substantially resistant to alcohol dose dumping” is meant that the extended-release (XR) particle(s) or region(s) of the single population of IR/XR-particles retains its extended-release characteristics even when exposed to high concentrations of ethanolic media or solution, for example, the release characteristics (release rate) of “viloxazine” from the extended-release (XR) particle(s) or region(s) of the single population of IR/XR-particles in an ethanol concentration of at least 4%, preferably 20%, more preferably 40% v/v in 900 ml of an aqueous medium (water, 0.1N HCl, acetate buffer pH 4.5, and/or phosphate buffer pH 6.8) is remain about similar to the release characteristics (release rate) of the “viloxazine” from the extended-release (XR) particle(s) or region(s) of the single population of IR/XR-particles in an ethanol-free environment, i.e., 900 ml of an aqueous medium (water, 0.1N HCl, acetate buffer pH 4.5, and/or phosphate buffer pH 6.8). Han Lennernas, “Ethanol-Drug Absorption Interaction: Potential for a Significant Effect on the Plasma Pharmacokinetics of Ethanol Vulnerable Formulations,” Mol. Pharmaceutics, 2009, 6 (5), pp 1429-1440, incorporated by reference in its entirety, recommends a two-hour time frame for screening the in vitro dissolution profile of an extended-release product in ethanol concentrations of up to 40% v/v. Therefore, one definition for “substantially resistant to alcohol dose dumping” is the extended-release (XR) particle(s) or region(s) of the single population of IR/XR-particles releases the viloxazine in an amount of less than about 75% of the total weight of the viloxazine contained in the extended-release (XR) particle(s) or region(s) in less than about 2 hours when measured in 900 ml of ethanolic aqueous media [contains up to 40% v/v ethanol in 900 ml of water, HCl, acetate buffer pH 4.5, or phosphate buffer pH 6.8; for example—mixture of 540 ml of HCl (60% v/v)+360 ml of ethanol (40% v/v) make 900 ml of 40% v/v ethanolic 0.1N HCl] at a temperature of 37°±1° C. using either USP apparatus 2 (paddle) at 50 RPM (or 75 RPM) or USP apparatus 1 (basket) at 100 RPM (or 150 RPM).

In certain embodiments, the release characteristics (release rate) of the viloxazine from the extended-release (XR) particle(s) or region(s) of the single population of IR/XR-particles are substantially resistant to alcohol dose dumping, wherein the extended-release (XR) particle(s) or region(s) of the single population of IR/XR-particles comprises a mixture of at least two release rate-controlling ingredient, wherein the at least one release rate-controlling ingredient is a water-insoluble ingredient and the at least another release rate-controlling ingredient is a water-soluble ingredient, wherein a % weight ratio of the water-insoluble ingredient to the water-soluble ingredient is any from about 20:80 to about 99.9:0.1, preferably from about 40:60 to about 99:1, more preferably from about 50:50 to about 96:4, and most preferable from about 60:40 to about respectively. However, the % weight ratio of the water-insoluble ingredient to the water-soluble ingredient varies depending on the selection of the grade of ingredient. For example, ethyl cellulose:guar gum (very low viscosity grade of guar gum) in a ratio of 90:10 is not effective to resist alcohol dose dumping, but ethyl cellulose:guar gum (high viscosity grade of the guar gum) in the same ratio of 90:10 at the same coating weight gain is highly effective to prevent alcohol dose dumping.

In certain embodiments, a pharmaceutical formulation formulated in a single population of IR/XR particles comprising viloxazine in an amount from 20% w/w to 80% w/w of the total weight of the pharmaceutical formulation; at least one release rate-controlling ingredient in an amount from 2% w/w to 80% w/w of the total weight of the pharmaceutical formulation; and, optionally, one or more pharmaceutically acceptable excipient,
wherein each IR/XR particle of the single population of IR/XR particles comprising an extended-release region (XR) of the viloxazine; and an immediate-release region (IR) of the viloxazine, wherein the immediate-release region (IR) is formed as coat surrounding the extended-release region (XR), wherein a weight ratio of the total weight of the extended-release region (XR) components and the total weight of the immediate-release region (IR) components is in a range of about 1.5:1 to about 50:1, respectively, wherein a weight ratio of the viloxazine present in the extended-release region (XR) and the immediate-release region (IR) is in a range of about 0.25:1 to about 25:1, respectively,
   wherein the extended-release region (XR) comprising the viloxazine; the at least one release rate-controlling ingredient; and optionally, the one or more pharmaceutically acceptable excipient; and
   wherein the immediate-release region (IR) comprising the viloxazine, and, optionally, the one or more pharmaceutically acceptable excipients.

In certain embodiments, a pharmaceutical formulation comprising a single population of IR/XR particles of viloxazine, wherein each IR/XR particle of the single population of IR/XR particles comprising an extended-release region (XR) of the viloxazine; and an immediate-release region (IR) of the viloxazine, wherein the immediate-release region (IR) is formed as coat surrounding the extended-release region (XR), wherein a weight ratio of the total weight of the extended-release region (XR) components and the total weight of the immediate-release region (IR) components is in a range of about 1.5:1 to about 50:1, respectively, wherein a weight ratio of the viloxazine present in the extended-release region (XR) and the immediate-release region (IR) is in a range of about 0.25:1 to about 25:1, respectively,
wherein the extended-release region (XR) comprising the viloxazine embedded within a matrix of the at least one release rate-controlling ingredient, wherein the matrix is in the form of a matrix particle or a matrix coat surrounding an inert core, wherein the matrix optionally coated with a release rate controlling coat comprising the at least one release rate-controlling ingredient; and
wherein the immediate-release region (IR) is formed as the immediate-release coat (IR) surrounding the extended-release region (XR), and comprising the viloxazine, and, optionally, the one or more pharmaceutically acceptable excipients.

In certain embodiments, a pharmaceutical formulation formulated in a single population of IR/XR particles comprising viloxazine in an amount from 20% w/w to 80% w/w of the total weight of the pharmaceutical formulation; at least one release rate-controlling ingredient in an amount from 20% w/w to 80% w/w of the total weight of the pharmaceutical formulation; and, optionally, one or more pharmaceutically acceptable excipient,
wherein each IR/XR particle of the single population of IR/XR particles comprising:
   (a) an extended-release particle (XR) of the viloxazine, wherein the extended-release particle (XR) comprising the viloxazine embedded within a matrix of the at least one release rate-controlling ingredient, wherein the matrix is in the form of a matrix particle or a matrix coat surrounding an inert core, wherein the matrix, optionally, further comprising the one or more pharmaceutically acceptable excipients, and is, optionally, further coated with a release rate controlling coat (RC) of the at least one release rate-controlling ingredient; and
   (b) an immediate-release coat (IR) surrounding the extended-release particle (XR), and comprising the viloxazine, and, optionally, the one or more pharmaceutically acceptable excipients,
   wherein a weight ratio of the total weight of the extended-release particle components and the total weight of the immediate-release coat (IR) components is in a range of about 1.5:1 to about 50:1, respectively,
   wherein a weight ratio of the viloxazine present in the extended-release particle (XR) and the immediate-release coat (IR) is in a range of about 0.25:1 to about 25:1, respectively.

In certain embodiments, a pharmaceutical formulation comprising a single population of IR/XR particles of viloxazine, wherein each IR/XR particle of the single population of IR/XR particles comprising an extended-release region (XR) of the viloxazine; and an immediate-release region (IR) of the viloxazine, wherein the immediate-release region (IR) is formed as coat surrounding the extended-release region (XR), wherein a weight ratio of the total weight of the extended-release region (XR) components and the total weight of the immediate-release region (IR) components is in a range of about 1.5:1 to about 50:1, respectively, wherein a weight ratio of the viloxazine present in the extended-release region (XR) and the immediate-release region (IR) is in a range of about 0.25:1 to about 25:1, respectively,
wherein the extended-release region (XR) comprising a release rate-controlling (RC) coat of the at least one release rate-controlling ingredient surrounding a core particle of the viloxazine, wherein at least one of the release rate-controlling (RC) and the core particle, optionally, further comprising one or more pharmaceutically acceptable excipients; and
wherein the immediate-release region (IR) is formed as the immediate-release coat (IR) surrounding the extended-release region (XR), and comprising the viloxazine, and, optionally, the one or more pharmaceutically acceptable excipients.

In certain embodiments, a pharmaceutical formulation formulated in a single population of IR/XR particles comprising viloxazine in an amount from 20% w/w to 80% w/w of the total weight of the pharmaceutical formulation; at least one release rate-controlling ingredient in an amount from 20% w/w to 80% w/w of the total weight of the pharmaceutical formulation; and, optionally, one or more pharmaceutically acceptable excipient,
wherein each IR/XR particle of the single population of IR/XR particles comprising:
   (a) an extended-release particle (XR) of the viloxazine, wherein the extended-release particle (XR) comprising a release rate-controlling (RC) coat of the at least one release rate-controlling ingredient surrounding a core particle of the viloxazine, wherein the release rate-controlling (RC) and the core particle, optionally, further comprising the one or more pharmaceutically acceptable excipients; and
   (b) an immediate-release coat (IR) surrounding the extended-release particle (XR), and comprising the viloxazine, and, optionally, the one or more pharmaceutically acceptable excipients,
   wherein a weight ratio of the total weight of the extended-release particle components and the total weight of the immediate-release coat (IR) components is in a range of about 1.5:1 to about 50:1, respectively,
   wherein a weight ratio of the viloxazine present in the extended-release particle (XR) and the immediate-release coat (IR) is in a range of about 0.25:1 to about 25:1, respectively.

In any of the previously discussed embodiments, the release rate-controlling (RC) coat comprises at least one release rate-controlling ingredient, and optionally, at least one or more pharmaceutically acceptable excipients.

In any of the previously discussed embodiments, the core particle of the viloxazine comprises the viloxazine, and optionally, at least one or more pharmaceutically acceptable excipients. In certain embodiments, the core particle of the viloxazine can be formulated or prepared or used in the form of (a) a particle of the viloxazine (a pure drug substance); (b) a matrix particle of the viloxazine, wherein the viloxazine is embedded within a matrix of the one or more pharmaceutically acceptable excipient; or (c) a drug coat of the viloxazine surrounding an inert core.

In any of the previously discussed embodiments, the inert core includes particle, bead, granule, or sphere of one or more pharmaceutically acceptable excipients, such as sugar sphere, microcrystalline cellulose sphere, silicon sphere, ion-exchange resin, placebo sphere such as in form of granules, beads, pellets, spheres, mini-tablets.

In certain embodiments, the weight ratio of the total weight of the extended-release region (XR) components (or XR-particle components) and the total weight of the immediate-release region (IR) components (IR coat components) is in a range of about 1.5:1 to about 50:1, preferably about 2:1 to about 40:1, more preferably about 3:1 to about 30:1, and most preferably about 5:1 to about 25:1, respectively.

In certain embodiments, the amount of the viloxazine contained in the immediate-release coat (or region) is not less than about 5%, preferably not less than about 15%, more preferably not less than about 35%, and most preferably not less than about 50% w/w of the total weight of the immediate-release coat (or region) components (drug and other pharmaceutically acceptable excipients if any).

In any of the previously discussed embodiments, an amount of the viloxazine contained in the extended-release (XR) particles (XR-particles) is not less than about 5%, preferably not less than about 15%, more preferably not less than about 35%, and most preferably not less than about 50% w/w of the total weight of the extended-release (XR) particles (XR-particles).

In certain embodiments, the overall amount of the viloxazine included in the single population of IR/XR particles of the pharmaceutical formulation is at least about 10 mg and a maximum of about 1000 mg. In certain embodiments, the weight ratio of the viloxazine present in the extended-release region (XR) and the immediate-release region (IR) of the single population of IR/XR particles of viloxazine is in a range of about 0.25:1 to about 25:1, respectively, wherein at least about 8 mg of the viloxazine is included in the immediate-release region (IR) of the viloxazine of the single population of IR/XR particles. In certain embodiments, the weight ratio of the viloxazine present in the extended-release region (XR) and the immediate-release region (IR) of the single population of IR/XR particles of viloxazine is in a range of about 0.25:1 to about respectively, wherein at least about 35 mg of the viloxazine is included in the extended-release region (XR) of the viloxazine of the single population of IR/XR particles.

In the present disclosure, the terms “material”, “excipient”, “additives”, and “ingredient” are interchangeable, and one of skill in the art is cognizant that these terms are open-ended terms.

In certain embodiments, the at least one release rate controlling ingredients is included in extended-release particulates (XR) or in the pharmaceutical formulation in an amount of less than about 75% w/w, from about 2.5% w/w to about 75% w/w, preferably from about 5% w/w to about 65% w/w, more preferably from about 5% w/w to about 60% w/w, most preferably from about 5% w/w to about 55% w/w or particularly preferably from about 5% w/w to about 50% w/w of total weight of the pharmaceutical formulation. The term “controlled release material”, “release rate controlling ingredient”, and “release retardant” are interchangeable. The term “release rate controlling ingredient” refers to any material that controls or extends the release rate of the viloxazine from the extended-release particles (XR-particles).

In certain embodiments, the at least one release rate-controlling ingredient includes at least one of water-insoluble material(s) and water-soluble material(s).

In certain embodiments, the term “water-insoluble material” refers to a component that is insoluble in water. The water-insoluble material includes wax, oil, and water-insoluble polymer. The suitable water-insoluble ingredient includes, but are not to be limited, cellulose derivatives include cellulose acetate, cellulose acetate butyrate, cellulose triacetate, ethyl cellulose; wax include microcrystalline wax, beeswax, glycowax, castor wax, carnauba wax, glycerol monostearate, glycerol palmitostearate; oil include hydrogenated vegetable oil, hydrogenated castor oil, vegetable oil, stearyl alcohol, acetylated hydrogenated soybean oil glycerides, castor oil; glycerol behenic acid ester, glyceryl behenate, glyceryl monooleate, glyceryl monostearate, propylene glycol monostearate, cetyl alcohol, natural and synthetic glycerides, fatty acids, fatty alcohol, lipid, methacrylic acid derivatives such as polymethacrylate and its copolymer (such as polymer under brand name Eudragit® such as eudragit RS, eudragit RL, eudragit NE, eudragit NM), PEG glyceryl esters, poly(ethyl acrylate-co-methyl methacrylate) ethyl acrylate methyl methacrylate copolymer, poly (ethyl acrylate-co-methyl methacrylate-cotrimethylammonioethyl methacrylate chloride), polyvinyl acetate, copolymers of vinyl pyrrolidone and vinyl acetate; vinyl acetate and copolymer thereof, ethyl vinyl acetate, modified starch like pregelatinised starch, polylactic acid or polyglycolic acid and copolymers thereof, methacrylates, cocoa butter, macrogol stearate, diethylene glycol monostearate, polyoxyethylene 50 stearate, and mixtures thereof.

The water-soluble material includes water-soluble gums, water-soluble polymers, polysaccharides, protein or polypeptides, sugar, acid, base, salt, starch or starch derivatives, and pH-sensitive water-soluble polymers.

The preferred water-soluble gums include, but are not limited to, xanthan gum, acacia gum, diutan gum, tragacanth, gellan gum, guar gum, fenugreek gum, locust bean gum, pullulan, welan gum, etc.

The preferred water-soluble polymer includes, but not limited to, cellulose derivatives such as carboxymethyl cellulose, cellulose ether (such as hydroxyethyl cellulose, hydroxypropylmethyl cellulose, hydroxypropyl cellulose, etc.), polyalkylene oxide and its co-polymer such as polyethylene oxide, copolymer of ethylene oxide-propylene oxide, polycarboxylic acid such as polyacrylic acid, polyolefinic alcohol (such as polyvinyl alcohol), or a polyvinyl lactam such as, e.g., polyvinylpyrrolidone, polyvinyl caprolactam, alginic acid and its derivative, methacrylic acid and its copolymer (such as polymer under brand name Eudragit®, polyacrylic acid and copolymer thereof (such as carbomer); protein or polypeptide such as gelatin, albumin, polylysine, soy protein; starch or its derivative; and like.

The polysaccharides are polymeric carbohydrate molecules composed of long chains of monosaccharide units bound together by glycosidic linkages and on hydrolysis give the constituent monosaccharides or oligosaccharides. They range in structure from linear to highly branched. Examples include storage polysaccharides such as starch and glycogen, and structural polysaccharides such as cellulose and chitin.

The preferred sugars include dextrose, glucose, arabinose, ribose, arabinose, xylose, lyxose, xylol, allose, altrose, inositol, glucose, sorbitol, mannose, gulose, glycerol, idose, galactose, talose, trehalose, mannitol, erythritol, ribitol, xylitol, maltitol, isomalt, lactitol, sucrose, raffinose, maltose, fructose, lactose, dextrin, dextran, amylase and xylan.

The preferred water-soluble salts include sodium chloride, potassium chloride, calcium chloride or magnesium chloride, lithium chloride, lithium, sodium or potassium hydrogen phosphate, lithium, sodium or potassium dihydrogen phosphate, salts of organic acids such as sodium or potassium acetate, sodium bicarbonate, magnesium succinate, sodium benzoate, sodium citrate or sodium ascorbate.

The preferred acids include ascorbic acid, 2-benzene carboxylic acid, benzoic acid, fumaric acid, citric acid, maleic acid, serbacic acid, sorbic acid, edipic acid, edetic acid, glutamic acid, toluene sulfonic acid, water-soluble amino acids such as glycine, leucine, alanine, or methionine and tartaric acid; and like.

The preferred pH-sensitive water-soluble polymer includes EUDRAGIT® FS 30 D (poly (methyl acrylate-co-methyl methacrylate-co-methacrylic acid)), EUDRAGIT® L 30 D-55, EUDRAGIT® L and EUDRAGIT® S (poly (methacrylic acid-co-methyl methacrylate)), hydroxypropyl methylcellulose acetate succinate, hydroxypropyl methylcellulose phthalate, cellulose acetate phthalate, shellac, zein, and combinations thereof.

In certain embodiments, the water-soluble materials can be used as a pore former or flux enhancer in the matrix or the release rate-controlling film.

In certain embodiments, the one or more pharmaceutically acceptable excipients, depending on their functionality in the pharmaceutical formulation components (extended-release particles and immediate-release coat) include, but are not limited to, filler, diluent, disintegrant, anti-tacking agent, binder, glidant, surfactant, wetting agent, lubricant, anti-oxidant, plasticizer, sweetener, coloring agent, sugar, salt, acid, and osmotic agent.

The preferred fillers or diluents include, but are not limited to, cellulose derivatives (such as microcrystalline cellulose), mono, di, or tri-basic calcium phosphate, sugar, carbohydrate, starch derivatives, acid or base, and like. Filler or diluent refers to material that is used to increase the bulk volume of a pharmaceutical formulation and/or to improve the content uniformity of the pharmaceutical formulation. The disintegrant may be highly/rapidly swellable, moderately swellable or slowly swellable such as vinylpyrrolidone polymers such as crospovidone, cellulose and cellulose derivatives, sodium starch glycolate, starch and starch derivatives, resins, and like. Suitable anti-tacking agent is selected from the group consisting of, but are not limited to, stearates, stearic acid, vegetable oil, waxes, a blend of magnesium stearate and sodium lauryl sulfate, boric acid, surfactants, sodium benzoate, sodium acetate, sodium chloride, DL-Leucine, polyethylene glycol, sodium oleate, sodium lauryl sulfate, magnesium lauryl sulfate, talc, corn starch, amorphous silicon dioxide, syloid, metallic stearates, Vitamin E, Vitamin E TPGS, silica and combinations thereof.

Suitable binder include, but are not to be limited, cellulose derivatives include, but are not limited to be, methylcellulose, hydroxypropyl methylcellulose, hydroxyethyl cellulose, hydroxypropyl cellulose, hydroxyethyl methylcellulose, ethyl hydroxyethylcellulose, ethyl methyl cellulose, hydroxymethyl cellulose, hydroxymethyl propyl cellulose, sodium carboxymethylcellulose, polyacrylamide derivatives, methacrylic acid derivatives, vinyl pyrrolidone polymers such as polyvinylpyrrolidone, a starch derivative, polyalkylene oxide and copolymer thereof, alkylene oxide homopolymers, gums of plant, animal, mineral or synthetic origin, polyacrylic acid and copolymer thereof, polyvinyl alcohols, polyethylene glycol, poloxamer, and mixtures thereof. Suitable glidants and lubricants may be incorporated such as stearic acid, metallic stearates, talc, waxes, glycerides with high melting temperatures, colloidal silica, sodium stearyl fumarate, polyethylene glycols, and alkyl sulfates. Suitable plasticizers include, but are not limited to, triacetin, triethyl acetate, acetylated monoglyceride, olive oil, acetyl tributyl citrate, acetyl triethyl citrate, glycerin, sorbitol, polyethylene glycol, polypropylene glycol and like. Stabilizers, such as antioxidants, to inhibit or retard oxidative drug decomposition during storage of the pharmaceutical composition. Natural or synthetic sweeteners include, but are not limited to, mannitol, sorbitol, saccharose, saccharine, aspartame, acesulfame K, or cyclamate. Preferred coloring agent includes D&C dye/lake, FD&C dye, an FD&C lake, caramel, ferric oxide, a natural coloring agent, and a combination thereof. The amount of coloring agent used will vary as desired. Preferably the composition is non-toxic, edible, stable in light and air and free of potential hazards to human health. The osmotic agent includes sugar and salts as described above.

Surfactants and Wetting agents include, but are not limited to heptadecaethylene oxycetanol, lecithins, sorbitol monooleate, polyoxyethylene sorbitol monooleate, polyoxyethylene stearate, polyoxyethylen sorbitan monolaurate, benzalkonium chloride, nonoxynol 10, oxtoxynol 9, polysorbates for example 20, 40, 60 or 80, sorbitan mono-palmitate, sodium salts of fatty alcohol-sulfates such as sodium lauryl sulfate, sodium dodecylsulfate, sodium salts of sulfosuccinates such as sodium dioctylsulfosuccinate, partially esters of fatty acids with alcohols such as glycerine monostearate, partially esters of fatty acids with sorbitans such as sorbitan monolaurate, partially esters of fatty acids with polyhydroxyethylene sorbitans such as polyethyleneglycol sorbitan monolaurate, -monostearate or -monooleate, ethers of fatty alcohols with polyhydroxyethylene, esters of fatty acids with polyhydroxyethylene, copolymers of ethylenoxide and propylenoxide (Pluronic®) and ethoxylated triglycerides.

Depending on the functionality, the tentative range for the concentration of pharmaceutically acceptable excipients to be used in the pharmaceutical formulation is 0.01% w/w to about 75% w/w of the total weight of the pharmaceutical formulation. Handbook of Pharmaceutical Excipients, 9^{th} Edition, Edited by Paul J Sheskey, Bruno C Hancock, Gary P Moss, and David J Goldfarb, which herein incorporated entirely, provides more information on the functionality and proportion of use for many pharmaceutical excipients to be used in the present disclosure.

The above-listed ingredients are for information only, which does not limit the scope of the present disclosure. Any ingredient that is used in pharmaceutical formulation and fulfills the objective of the subject matter of the present disclosure is within the scope of the present disclosure.

In certain embodiments, a process for making a single population of IR/XR particles of viloxazine comprising the steps in sequence of:
a) the first step is forming an extended-release (XR) region(s) or particles of the viloxazine, and
b) the second step is forming an immediate-release (IR) region(s) or coat of the viloxazine surrounding the extended-release (XR) region(s) or particles of the viloxazine.

In certain embodiments, each extended-release particle (XR-particle) or region of the single population of IR/XR particles of viloxazine is prepared by embedding or dispersing the viloxazine within a matrix of the at least one release rate-controlling ingredient, wherein the matrix is prepared in the form of a matrix particle or a matrix coat. In certain embodiments, a mixture of the viloxazine with the at least one release rate-controlling ingredient forms the matrix particles or the matrix coats that are capable of limiting the dissolution rate of the viloxazine from the extended-release particle (XR-particle) into an aqueous medium. The matrix material (such as the at least one release rate-controlling ingredient) useful for these embodiments are generally water-insoluble materials, slowly water-soluble material, or a combination of water-insoluble materials and water-soluble materials. If needed, the matrix material(s) may optionally be formulated with water-soluble materials, which can be used as binders or as permeability-modifying agents in the matrix. In certain embodiments, the prepared XR-particles are further coated with an optional release rate controlling coat, wherein the release rate controlling coat comprises water-insoluble materials, slowly water-soluble material, or a combination of water-insoluble materials, and water-soluble materials.

In certain embodiments, each extended-release particle (XR-particle) or region of the single population of IR/XR particles of viloxazine is prepared by applying a release rate-controlling (RC) coat of the at least one release rate-controlling ingredient surrounding a core particle of the viloxazine, wherein the release rate-controlling (RC) coat, optionally, further comprising the one or more pharmaceutically acceptable excipients. In certain embodiments, the applied release rate-controlling (RC) coat surrounding the core particles of the viloxazine is capable of limiting the dissolution rate of the viloxazine from the extended-release particles (XR-particles) into an aqueous medium. The release rate-controlling ingredients useful to prepare the release rate-controlling coat are generally water-insoluble materials, slowly water-soluble materials, or a combination of water-insoluble materials and water-soluble materials. If needed, the water-soluble materials in an appropriate amount can be added as permeability-modifying agents in the release rate-controlling coat to achieve the desired extended-release profile of viloxazine. In certain embodiments, the viloxazine can also be used as a soluble material or permeability-modifying agent in the release rate controlling coat to achieve the desired extended-release profile of viloxazine. In certain embodiments, the prepared XR-particles are, optionally, further coated with a second release rate controlling coat, wherein the second release rate controlling coat comprises water-insoluble materials, slowly water-soluble material, or a combination of water-insoluble materials, and water-soluble materials. In certain embodiments, the core particle of the viloxazine comprises the viloxazine, and optionally, at least one or more pharmaceutically acceptable excipients, wherein the core particle of the viloxazine can be prepared, formulated or used in the form of (a) a particle of the viloxazine (a pure drug substance); (b) a matrix particle of the viloxazine, wherein the viloxazine is embedded within a matrix of the one or more pharmaceutically acceptable excipient; or (c) a coat of the viloxazine surrounding an inert core.

In any of the previously discussed embodiments, the viloxazine is embedded within a matrix of the at least one release rate-controlling ingredient and/or one or more pharmaceutically acceptable excipients to form a matrix particle. A preferred process for manufacturing matrix particles is the extrusion/spheronization process. For this process, the viloxazine is wet-massed with matrix material [such as the at least one release rate-controlling ingredient and/or one or more pharmaceutically acceptable excipients (such as binder)], extruded through a perforated plate or die, and placed on a rotating disk. The extrudate ideally breaks into pieces which are rounded into spheres, spheroids, or rounded rods on the rotating plate. Another preferred process for manufacturing matrix particles is compressing blend mixture of viloxazine and matrix material [such as the at least one release rate-controlling ingredient and/or the one or more pharmaceutically acceptable excipients] to form mini-tablets. A further preferred process for manufacturing matrix particles is the preparation of melt granules. In this process, a desired amount of viloxazine is stirred with matrix material having a low melting point at an elevated temperature or above the melting temperature of the matrix material to form a homogeneous mixture, cooled, and then forced through a screen to form granules. Melt granules can be prepared by applying heat while mixing viloxazine with matrix material having a low melting point in a granulator such as a rapid mixture granulator. A further preferred process for manufacturing matrix particles involves using an organic solvent to aid in the mixing of the viloxazine with the matrix material. This technique can be used when it is desired to utilize a matrix material with an unsuitably high melting point that, if the material were employed in a molten state, would cause decomposition of the drug or of the matrix material, or would result in an unacceptable melt viscosity, thereby preventing mixing of viloxazine with the matrix material. Viloxazine and matrix material may be combined with a modest amount of solvent to form a paste and then forced through a screen to form granules from which the solvent is then removed. Alternatively, viloxazine and matrix material may be combined with enough solvent to completely dissolve the matrix material with the drug, and the resulting solution spray dried to form the matrix particulates or particles.

In certain embodiments, the viloxazine is embedded within a matrix of the at least one release rate-controlling ingredient and/or one or more pharmaceutical-acceptable excipients to form a matrix coat or a drug coat surrounding an inert core. A most preferred process for manufacturing the matrix coat or the drug coat is: dissolve the viloxazine and the matrix material (the at least one release rate-controlling ingredient and/or one or more pharmaceutical-acceptable excipients) in enough amount of solvent (aqueous, organic, or mixture thereof), and spray the resulting solution on inert cores (particle, bead, granule, or sphere of one or more pharmaceutically acceptable excipients, such as sugar sphere, microcrystalline cellulose sphere, silicon sphere, ion-exchange resin, placebo sphere such as in form of granules, beads, pellets, spheres, mini-tablets) to form the matrix coat or the drug coat surrounding the inert cores. The conventional pan coating or Wurster coating process can be used to prepare the matrix coat or the drug coat surrounding the inert core. A further useful process for preparing the matrix coat or the drug coat is powder layering of the mixture of the matrix material and the viloxazine surrounding the inert core.

In certain embodiments, the drug coat of viloxazine surrounding the inert coat comprises only viloxazine (free from any pharmaceutically acceptable excipient).

In any of the previously discussed embodiments, the inert core includes particle, bead, granule, or sphere of one or more pharmaceutically acceptable excipients, such as sugar sphere, microcrystalline cellulose sphere, silicon sphere, ion-exchange resin, placebo sphere such as in form of granules, beads, pellets, spheres, mini-tablets.

In certain embodiments, the immediate release coat or region of viloxazine is included surrounding each XR-particle of the single population of IR/XR particles of viloxazine. A most preferred process for manufacturing the immediate release coat or region of viloxazine is: dissolve the viloxazine, and optionally, one or more pharmaceutical-acceptable excipients in enough amount of solvent (aqueous, organic, or mixture thereof), and spray the resulting solution on the prepared XR-particles of viloxazine to form the immediate release coat or region of viloxazine surrounding each XR-particle of the single population of IR/XR particles of viloxazine. The conventional pan coating or Wurster coating process can be used to prepare the immediate release coat or region of viloxazine. A further useful process for preparing the immediate release coat or region of viloxazine is powder layering of the viloxazine or a mixture of the viloxazine and one or more pharmaceutical-acceptable excipients surrounding the XR-particles of viloxazine to form the immediate release coat or region of viloxazine surrounding each XR-particle of the single population of IR/XR particles of viloxazine.

The various coating processes that may be useful to produce different coats [such as a coat of the viloxazine surrounding an inert core, an immediate-release coat (IR) of the viloxazine surrounding the extended-release particle (XR), a release rate-controlling (RC) coat of the at least one release rate-controlling ingredient surrounding a core particle of the viloxazine, an optional release rate controlling coat (RC) of the at least one release rate-controlling ingredient surrounding the extended-release particle (XR) of viloxazine or any other type of coat] includes solution spraying, dry powder layering, compression coating, hot melt coating, supercritical fluid coating, electrostatic spray coating, etc.

Various process useful for manufacturing the presently disclosed formulation includes granulation, palletization, roller compaction, compression, wet granulation, Wurster coating, fluid bed granulation, rotary bed granulation, forming complexation of drug with ion-exchange resin, wet granulation with extrusion and spherization, hot melt extrusion, injection molding, conventional pan coating, roller compaction, etc.

Once the single population of IR/XR particles of viloxazine are formed, then the IR/XR particles may be blended with compressible excipients and then compressed the blend to form a tablet. Once the single population of IR/XR particles of viloxazine is formed, then the IR/XR particles may be filled in a capsule or sachet or can be mixed with pharmaceutically acceptable excipients to form a powder for suspension. The single population of IR/XR particles of viloxazine can be administered after sprinkling on food or juice.

The presently disclosed pharmaceutical formulation is not limited to the art of fabricating IR/XR-particles of viloxazine, in other word, an ordinary skill in this art can also manufactured IR/XR-particles of viloxazine by any other method than described herein.

Another aspect of the present disclosure is the treatment of CNS disorders in mammalian subjects, including but not limited to the treatment of ADHD, ADHD-related disorders, and depressive disorders, with the presently disclosed extended-release formulations of viloxazine as described herein. One treatment regimen comprises administering the viloxazine formulation of the present disclosure to a subject once or twice a day to provide a total daily dose ranging from 10 mg to 1000 mg of viloxazine (base).

Another aspect of the present disclosure is to reduce the overall weight of the pharmaceutical formulation and overall manufacturing time by eliminating the requirement of additional manufacturing procedures and unit operation such as preparing separate particles of immediate-release viloxazine.

In an additional embodiment, the present subject matter also discloses formulations of viloxazine that can provide therapeutic levels of the drug for the period of time from 4 to 24 hours, or for the periods of from 4 to 20 hours, or for the periods of from 6 to 16 hours. Further, the formulations of the present subject matter are characterized by a maximum steady-state plasma concentration (Cₘₐₓ) of viloxazine which is higher than the minimal therapeutically effective concentration and is in the range of 50% to 125% relative to the maximum plasma concentration produced by the administration of from 50 mg to 800 mg, in particular, of from 100 mg to 800 mg, of from 100 mg to 650 mg, of from 100 mg to 500 mg of viloxazine as an IR formulation three times daily (TID) or twice daily (BID).

In certain embodiments, compared to the QELBREE™ (viloxazine extended-release capsule), the presently disclosed pharmaceutical formulations release viloxazine in an amount less than about 75% w/w of the total amount of viloxazine contained in the pharmaceutical formulation in less than about 2 hours when measured in 900 ml of 40% v/v ethanolic aqueous media [contains 40% v/v ethanol in 900 ml of 0.1N HCl or phosphate buffer pH 6.8] at a temperature of 37°±1° C. using either USP apparatus 2 (paddle) at 50 RPM (or 75 RPM) or USP apparatus 1 (basket) at 100 RPM (or 150 RPM).

In certain embodiments, the presently disclosed pharmaceutical formulations after administering with the 40% alcoholic beverages (e.g., 40% alcohol in orange juice) provide a relative mean or median Tₘₐₓ of viloxazine in the range of about 50% to about 200% as compared to administering the same dose of the presently disclosed pharmaceutical formulations without the 40% alcoholic beverages (eg. 0% alcohol in orange juice).

In certain embodiments, the presently disclosed pharmaceutical formulations after administering with the 40% alcoholic beverages (e.g., 40% alcohol in orange juice) provide at least about 50% higher relative mean or median Tₘₐₓ of viloxazine compared to administering the same dose of the QELBREE™ (viloxazine extended-release capsule) with the 40% alcoholic beverages (e.g., 40% alcohol in orange juice).

In one embodiment, the presently disclosed formulations provide for a relative mean or median Cₘₐₓ in the range of 80% to 125%, as compared to QELBREE™ (viloxazine extended-release capsule). In the other embodiment, the presently disclosed pharmaceutical formulation provides the Cₘₐₓ which is lower than the maximum plasma concentration produced by viloxazine administered as an same amount IR formulation of viloxazine TID or BID. The presently disclosed formulations also provide a relative steady-state area under the viloxazine plasma concentration-time profiles for a 24-hour dosing interval (AUCₜₐᵤ) in the range of 80% to 125% as compared to QELBREE™ (viloxazine extended-release capsule).

In a further embodiment, the present subject matter discloses a method of treatment of the CNS disorders such as listed above with a high drug load formulation of viloxazine exhibiting an extended-release profile.

In certain embodiments, for administration to the mammal by the non-parenteral route such as oral, the presently disclosed pharmaceutical formulation can be compressed (with other one or more pharmaceutically acceptable excipients) to form a tablet, fill in a capsule to form a capsule dosage form, prepared as particles, prepared in suspension or a unit dose packet (sometimes refer to in the art as a “sachet”). In certain embodiments, the presently disclosed pharmaceutical formulation can be used as a sprinkle formulation, wherein the single population of IR/XR-particles can be sprinkled onto soft food or in a liquid, which is then swallowed.

### EXAMPLES

The invention will now be illustrated by the following examples which are not to be taken as limiting. In general, the examples demonstrate the formulation and process preparation of the presently disclosed pharmaceutical formulation formulated in single population of IR/XR-particles of viloxazine within the scope of this invention.

### Example 1: The Formula for a Single Population of IR/XR-Particles of Viloxazine

| | | | |
|---|---|---|---|
| Sr. | | Formulation 1 | Formulation 2** |
| No. | Ingredients | (% w/w) | (% w/w) |

XR-particles Components: Core particles

| | | | |
|---|---|---|---|
| 1 | Viloxazine Hydrochloride | 160.0 | 160.0 |
| 2 | Hypromellose 5 cps | 20.0 | 20.0 |
| 3 | Sugar spheres | 300.0 | 300.0 |
| 4 | Water | q.s. | q.s. |

XR-particles Components: Seal Coat

| | | | |
|---|---|---|---|
| 5 | Opadry ® (HPMC based) | 15.0 | 15.0 |
| 6 | Water | q.s. | q.s. |

XR-particles Components: Release Rate Controlling Coat

| | | | |
|---|---|---|---|
| 7 | Aquacoat ECD 30 dispersion* (alcohol soluble - yes) | 183.3 | — |
| | Surelease ® E-7-19040* (alcohol soluble - yes) | — | 248.4 |
| 8 | Dibutyl Sebacate | 18.45 | — |
| 9 | Guar Gum (alcohol soluble - no) | 10.55 | — |
| | Hypromellose 5 cps (alcohol soluble - no) | — | 6.9 |
| 10 | Water | q.s. | q.s. |

Immediate-release Coat Components

| | | | |
|---|---|---|---|
| 11 | Viloxazine Hydrochloride | 40.00 | 40.00 |
| 12 | Hypromellose 5cps | 10.00 | 10.00 |
| 13 | Water | q.s. | q.s. |

| | | |
|---|---|---|
| In-Vitro Alcohol Dose-Dumping Sensitivity in 40% v/v | Very Low | Very High |
| ethanolic dissolution media | | |

*Aquacoat ECD 30 and Surelease ® E-7-19040 is ethyl cellulose dispersions containing 30% w/w and contains 25% w/w solid content, respectively. Ethyl cellulose is alcohol soluble.
**Formulation 2 contains similar extended-release components to the prior art US9662338B2, US9603853B2, and US9358204B2, and QELBREE ™ (viloxazine extended-release capsule).

The weight ratio of the total weight of XR-particles components and the total weight of IR coat components is 14.82:1 [741.0 mg (total weight of XR-particles components)/50.0 mg (total weight of IR coat components)].

The weight ratio of the viloxazine present in the extended-release particle (XR) and the immediate-release coat (IR) is 4:1 [160 (weight of viloxazine in the extended-release particle (XR)/40 (weight of viloxazine in the immediate-release (IR) coat)].

The amount of the viloxazine contained in the immediate-release coat (or region) is 80% w/w of the total weight of the immediate-release coat components [40 (amount of viloxazine in the immediate-release coat)/50 (total weight of IR coat components)].

Manufacturing Procedures:
a) Preparation of XR-particles components: Preparation of Core Particles of Viloxazine—
   i) Add the viloxazine hydrochloride followed by Hypromellose 5 cps in water (20% w/w solid content) to prepare the dispersion of viloxazine HCl.
   ii) Transfer the sugar spheres into FBP, then apply the step [i)] viloxazine dispersion on the sugar spheres using the appropriate coating parameter to produce a drug coat of viloxazine on the sugar spheres, thereby producing core particles of viloxazine.
b) Final XR-Particles components:
   i) Add Dibutyl Sebacate in Aquacoat ECD 30 and stir it for at least 2 hours.
   ii) Dissolve the guar gum or Hypromellose 5 cps in appropriate amount of water.
   iii) Add the step [b) ii)] solution into step [b) i)] Aquacoat ECD 30 dispersion and stir it for 45 minutes.
   iv) Apply the step [b) iii)] prepared dispersion on the step [a) ii)] core particles of viloxazine using appropriate coating parameter to produce a release rate controlling coat on the core particles of viloxazine, thereby producing XR-particles of viloxazine.
c) Preparation of Immediate Release Coat of Viloxazine on XR-particles
   i) Add the viloxazine hydrochloride followed by Hypromellose 5 cps in water (20% w/w solid content) to prepare the dispersion of viloxazine HCl.
   ii) Apply the step [c) i)] prepared dispersion on the step [b) iv)] XR-particles of viloxazine using appropriate coating parameter to produce immediate release coat of viloxazine on the XR-particles, thereby produced IR/XR-particles of viloxazine.

What is claimed is:

1. A pharmaceutical formulation comprising a single population of IR/XR particles of viloxazine,
wherein each IR/XR particle of the single population of IR/XR particles comprising an extended-release region (XR) of the viloxazine; and an immediate-release region (IR) of the viloxazine, wherein the immediate-release region (IR) of the viloxazine is coated surrounding the extended-release region (XR) of the viloxazine,
wherein the extended-release region (XR) is formed as an extended-release particle,
wherein the extended-release particle comprising
the viloxazine that is embedded within a matrix of at least one release rate-controlling ingredient, wherein the matrix is in the form of a matrix particle or a matrix coat surrounding an inert core, wherein the matrix, optionally, further comprising one or more pharmaceutically acceptable excipients, and is, optionally, further coated with a release rate controlling coat (RC) of the at least one release rate-controlling ingredient;
or
a release rate-controlling (RC) coat of at least one release rate-controlling ingredient surrounding a core particle of the viloxazine, wherein at least one of the release rate-controlling coat (RC) and the core particle, optionally, further comprising one or more pharmaceutically acceptable excipients,
wherein the immediate-release region (IR) is formed as a coat surrounding the extended-release particle, and comprising the viloxazine; and, optionally, the one or more pharmaceutically acceptable excipients,
wherein a weight ratio of the total weight of the extended-release region (XR) components and the total weight of the immediate-release region (IR) components present in the single population of IR/XR particles of viloxazine is in a range of about 1.5:1 to about 19:1, respectively,
wherein a weight ratio of the viloxazine present in the extended-release region (XR) and the immediate-release region (IR) of the single population of IR/XR particles of viloxazine is in a range of about 1.5:1 to about 19:1, respectively, wherein the amount of the viloxazine contained in the immediate-release region is not less than about 50% w/w of the total weight of the immediate-release region (IR) components,
wherein a process for making the single population of IR/XR particles of viloxazine comprising the steps in the sequence of:
(a) making the extended-release (XR) particles of the viloxazine; and
(b) making the immediate-release (IR) coat of the viloxazine surrounding the extended-release (XR) particles of the viloxazine.

2. The pharmaceutical formulation of claim 1, wherein the single population of IR/XR particles comprising the viloxazine from 20 mg to 855 mg, wherein the viloxazine in an amount from about 8 mg to about 180 mg is included in the immediate-release region (IR) of the single population of IR/XR particles.

3. The pharmaceutical formulation of claim 1, wherein the single population of IR/XR particles comprising the viloxazine from 20 mg to 855 mg, wherein the viloxazine in an amount from about 35 mg to 675 mg is included in the extended-release region (IR) of the single population of IR/XR particles.

4. The pharmaceutical formulation of claim 1, wherein the release of the viloxazine from the extended-release (XR) region of the single population of IR/XR-particles is substantially resistant to alcohol dose dumping when measured in 900 ml of an aqueous medium comprising up to 40% v/v ethanol, at a temperature of 37°±1° C., using either USP apparatus 2 (paddle) at RPM or USP apparatus 1 (basket) at 100 RPM.

5. The pharmaceutical formulation of claim 1 after administering with 40% alcoholic beverages provides a relative mean or median Tₘₐₓ of viloxazine in the range of 50% to 200%, as compared to administering the same dose of the pharmaceutical formulations with the non-alcoholic beverages.

6. The pharmaceutical formulation of claim 1 after administering with 40% alcoholic beverages provides at least about 50% higher relative mean or median Tₘₐₓ of viloxazine, as compared to administering the same dose of the QELBREE™ (viloxazine extended-release capsule) with the 40% alcoholic beverages.

7. The pharmaceutical formulation of claim 1, wherein the immediate-release region (IR) of the single population of IR/XR particles releases the viloxazine in an amount of more than about 75% of the total weight of the viloxazine contained in the immediate-release region of the single population of IR/XR particles in less than about 1 hour when measured in 900 ml of an aqueous medium having pH 6.8, at a temperature of 37°±1° C., using either USP apparatus 2 (paddle) at 50 RPM or USP apparatus 1 (basket) at 100 RPM.

8. The pharmaceutical formulation of claim 1, wherein the extended-release region (XR) of the single population of IR/XR particles releases the viloxazine in an amount of less than about 75% of the total weight of the viloxazine contained in the extended-release region of the single population of IR/XR particles in less than about 2 hours when measured in 900 ml of an aqueous medium having pH 6.8, at a temperature of 37°±1° C., using either USP apparatus 2 (paddle) at RPM or USP apparatus 1 (basket) at 100 RPM.

9. The pharmaceutical formulation of claim 1, wherein the matrix is in the form of the matrix coat surrounding the inert core, wherein the inert core includes at least one selected from particle; bead; granule; sphere of one or more pharmaceutically acceptable excipients including at least one of sugar sphere; microcrystalline cellulose sphere; silicon sphere; ion-exchange resin; or placebo sphere in form of at least one of granule; bead; pellet; sphere; mini-tablet.

10. The pharmaceutical formulation of claim 1, wherein the extended-release particle comprising the release rate-controlling (RC) coat of the at least one release rate-controlling ingredient surrounding the core particle of the viloxazine, wherein the core particle of the viloxazine is in the form of (a) a particle of the viloxazine as a pure drug substance; (b) a matrix particle of the viloxazine, wherein the viloxazine is embedded within a matrix of the one or more pharmaceutically acceptable excipient; or (c) a drug coat of the viloxazine surrounding an inert core, wherein the drug coat of the viloxazine comprising the viloxazine and, optionally, the one or more pharmaceutically acceptable excipients, and the inert core includes at least one selected from particle; bead; granule; sphere of one or more pharmaceutically acceptable excipients including at least one of sugar sphere; microcrystalline cellulose sphere; silicon sphere; ion-exchange resin; or placebo sphere in form of at least one of granule; bead; pellet; sphere; mini-tablet.

11. The pharmaceutical formulation of claim 1, wherein an amount of the at least one release rate-controlling ingredient included in the extended-release region (XR) of the single population of IR/XR particles is from about 2.5% w/w to about 75% w/w of the total weight of the pharmaceutical formulation.

12. The pharmaceutical formulation of claim 1, wherein the at least one release rate-controlling ingredient included in the extended-release region (XR) of the single population of IR/XR particles is at least one selected from cellulose acetate; cellulose acetate butyrate; cellulose triacetate; cellulose acetate propionate; ethyl cellulose; wax; glycerol monostearate; glycerol palmitostearate; glyceryl behenate; hydrogenated vegetable oil; hydrogenated castor oil; stearyl alcohol; glyceryl monostearate; propylene glycol monostearate; cetyl alcohol; methacrylic acid derivatives; polyvinyl acetate; copolymers of vinyl pyrrolidone and vinyl acetate; ethyl vinyl acetate; polylactic acid; polyglycolic acid and copolymers thereof; coco a butter; macrogol stearate; diethylene glycol monostearate; PEG glyceryl esters; poly(ethyl acrylate-co-methyl methacrylate) ethyl acrylate methyl methacrylate copolymer; poly (ethyl acrylate-co-methyl methacrylate-cotrimethylammonioethyl methacrylate chloride); polyoxyethylene 50 stearate; or any combination thereof.

13. The pharmaceutical formulation of claim 1, wherein each extended-release particle of the single population of IR/XR particles comprising at least two release rate-controlling ingredients, wherein the at least one release rate-controlling ingredient is a water-insoluble ingredient, and the at least another release rate-controlling ingredient is a water-soluble ingredient, wherein a % weight ratio of the water-insoluble ingredient to the water-soluble ingredient is in a range of about 35:65 to about 99:1, respectively.

14. The pharmaceutical formulation of claim 13, wherein the water-insoluble ingredient is at least one selected from cellulose acetate; cellulose acetate butyrate; cellulose triacetate; cellulose acetate propionate; ethyl cellulose; wax; glycerol monostearate; glycerol palmitostearate; glyceryl behenate; hydrogenated vegetable oil; hydrogenated castor oil; stearyl alcohol; glyceryl monostearate; propylene glycol monostearate; cetyl alcohol; methacrylic acid derivatives; polyvinyl acetate; copolymers of vinyl pyrrolidone and vinyl acetate; ethyl vinyl acetate; polylactic acid; polyglycolic acid and copolymers thereof; cocoa butter; macrogol stearate; diethylene glycol monostearate; PEG glyceryl esters; poly(ethyl acrylate-co-methyl methacrylate) ethyl acrylate methyl methacrylate copolymer; poly (ethyl acrylate-co-methyl methacrylate-cotrimethylammonioethyl methacrylate chloride); polyoxyethylene 50 stearate; or any combination thereof.

15. The pharmaceutical formulation of claim 13, wherein the water-soluble ingredient is at least one selected from xanthan gum; acacia gum; diutan gum; tragacanth; gellan gum; guar gum; fenugreek gum; locust bean gum; pullulan; welan gum; sodium carboxymethyl cellulose; cellulose ether derivatives including hydroxyethyl cellulose, hydroxypropylmethyl cellulose, hydroxypropyl cellulose; polyalkylene oxide and its co-polymer including polyethylene oxide; copolymer of ethylene oxide-propylene oxide; carbopol; polyvinyl alcohol; polyvinyl pyrrolidone; sodium alginate; polysaccharide; starch and starch derivative; dextrose; glucose; arabinose; ribose; arabinose; xylose; lyxose; xylol; allose; altrose; inositol; glucose; sorbitol; mannose; gulose; glycerol; idose; galactose; talose; trehalose; mannitol; erythritol; ribitol; xylitol; maltitol; isomalt; lactitol; sucrose; raffinose; maltose; fructose; lactose; dextrin; dextran; amylase; xylan; sodium chloride; potassium chloride; calcium chloride; magnesium chloride; lithium chloride; sodium or potassium hydrogen phosphate; sodium or potassium dihydrogen phosphate; salts of organic acids including sodium or potassium acetate, sodium bicarbonate, magnesium succinate, sodium benzoate, sodium citrate; sodium ascorbate; ascorbic acid; 2-benzene carboxylic acid; benzoic acid; fumaric acid; citric acid; maleic acid; serbacic acid; sorbic acid; edipic acid; edetic acid; glutamic acid; toluene sulfonic acid; and water-soluble amino acids.

16. The pharmaceutical formulation of claim 1 is in a dosage form selected from tablets, capsules, beads, granules, powders, caplets, troches, sachets, cachets, pouches, and sprinkles, wherein the dosage form is suitable for oral administration.

17. The pharmaceutical formulation of claim 1, wherein the viloxazine is in the form of viloxazine hydrochloride.

18. The pharmaceutical formulation of claim 1, wherein the single population of IR/XR particles of the pharmaceutical formulation comprising the viloxazine hydrochloride from 20 mg to 855 mg when administered orally by a patient, provides treatment in at least one therapeutic condition selected from ADHD, ADHD-related disorders, and depressive disorders.

19. The pharmaceutical formulation of claim 18 is suitable for once-a-day administration to provide the treatment in at least one therapeutic condition selected from ADHD, ADHD-related disorders, and depressive disorders.

20. The pharmaceutical formulation of claim 18 is suitable for twice-a-day administration to provide the treatment in at least one therapeutic condition selected from ADHD, ADHD-related disorders, and depressive disorders.
